(19) United States
(12) Patent Application Publication
(10) Pub. No.: US 2003/0195526 A1
(43) Pub. Date: Oct. 16, 2003

(54) Marker for an instrument and methods for localizing a marker

(76) Inventor: Stefan Vilsmeier, Kufstein (AT)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 10/142,269

(22) Filed: May 9, 2002

(30) Foreign Application Priority Data
Apr. 16, 2002 (EP) 02 008 185.7

(51) Int. Cl. A61B 19/00

(52) U.S. Cl. 606/130

(57) ABSTRACT

The invention relates to a marker for an instrument, wherein the marker can be arranged on at least an area of the instrument, along a surface encircling the instrument, and to a method for iteratively localizing at least one marker on an instrument, wherein the position of the reflection center of at least two markers is detected, from which a correction value for determining the spatial position of an instrument connected to the at least two markers is determined.

The present invention relates to a marker for an instrument, preferably for a rotationally symmetrical medical instrument, to a system which uses such markers, and to methods for localizing at least one marker. A marker in accordance with the invention can be used to localize and/or navigate an instrument, in particular in “image guided surgery”. In general, the invention can be used in microsurgery, orthopaedics, spinal or cranial surgery or in other areas.

For navigating or localizing instruments, such as for example medical instruments, a known method is to attach a so-called reference star to the medical instrument, at whose ends markers—for example active, light-emitting elements or passive, reflective surfaces—are arranged. A camera which detects the light emitted or reflected by the markers can provide information from which the spatial position of the markers and therefore the position of the instrument connected to the markers and/or the reference star can be determined. In this way, an instrument can be navigated, i.e. on the basis of the spatial position of the instrument thus determined, the instrument can be precisely moved by computer assistance to a desired location, e.g. to a particular point of a body, which is preferably likewise connected to markers.

If, for example, the instrument exhibits an axis which rotates while the instrument is being used, such as for example a screwdriver or a drill, then it is not advantageous to simply attach markers to this axis, since the rotation moves them out of the visual range of a camera. In order to solve this problem, it has already been proposed to attach a bearing to the rotating instrument, to which the markers can be attached. However, when using the rotating instrument, an operator constantly has to take care that the markers attached to the bearing are held in the direction of the camera, which makes handling the instrument more difficult.

Furthermore, a bearing means an additional structural requirement which precipitates higher production costs and greater susceptibility to faults. Similarly, a relatively large effort is required to sterilize a device having such a structurally elaborate design.

Methods and devices for localizing and navigating instruments are known from DE 196 39 615 A1, DE 195 36 180 A1, DE 100 00 937 A1 and DE 296 23 941 U1 of the Applicant.

It is an object of the present invention to propose a marker for an instrument, a system which uses such a marker, and methods for localizing at least one marker, with which instruments—such as for example rotationally symmetrical instruments—can easily be localized.

This object is solved by the invention defined in the independent claims. Advantageous embodiments follow from the dependent claims.

A marker in accordance with the invention, which is preferably used for instruments which are moved or rotated when used, in particular instruments comprising an axis of rotation or instruments comprising a rotationally symmetrical area, is realized in accordance with the invention in such a way that it can be attached to at least an area of the instrument, along at least a part of a surface area encircling the instrument. The marker in accordance with the invention can for example be a band, a number of band sections or a continuous ring or individual ring sections, encircling an instrument, for example a cylindrical object, with for example an approximately constant width from the surface, such that the cylindrical object can be rotated about its longitudinal axis, wherein irrespective of the rotational angle of the cylindrical object, at least a partial area of the encircling ring or band can always be detected by a camera, as long as the camera is not positioned on a direction extending along the longitudinal axis of the cylindrical object but can detect a lateral area of the cylindrical object.

The marker in accordance with the invention can consist of one continuous element or alternatively can also consist of a number of individual elements which can be arranged in a distribution about the surface of an instrument such that, irrespective of the rotational direction of the instrument, at least one part of the marker or one individual element can be detected by a camera arranged laterally with respect to the instrument. A ring, for example, can be used, which is discontinuous at particular parts of its circumference, or various individual light-emitting or light-reflecting surfaces having and approximately identical or also different geometry can be attached around the surface of the instrument such that, irrespective of the rotational direction of the instrument, at least one marker element can be seen in a lateral view of the instrument.

In general, it should be possible to arrange a marker in accordance with the invention, which can consist of one continuous or of a number of individual discrete elements, distributed about at least a partial area of the surface of an instrument such that, irrespective of the orientation or rotation of the instrument, at least a partial area of the marker or one partial element of the marker can be detected by a camera which is substantially not slaved by a movement of the instrument. Such an arrangement of markers also facilitates sterilization, since smooth surfaces are simpler and easier to disinfect than markers attached to a bearing on a reference star.

In general terms, a marker or a partial element of a marker in accordance with the invention is an element which can emit light. This can, for example, be achieved actively using LEDs or other light-emitting elements, or reflective surfaces can be used which can reflect light which hits the marker from without. This can, for example, be visible light or infrared radiation, which is then detected by at least one camera.

The marker in accordance with the invention is preferably an approximately rotationally symmetrical element, for example a ring-shaped or band-shaped element can be slid along the longitudinal axis and preferably fixed onto an instrument, preferably an approximately rotationally symmetrical element, or for example can be laterally clipped or locked onto the instrument, or for example arranged encircling on the instrument as an adhesive band having a reflective surface.

It is further possible for example to design the marker to be spherical or conical, wherein said spherical or conical element, having a reflective surface, can be arranged around the instrument such that when the instrument is shifted or rotated, at least a partial area of the surface can be detected by a camera arranged laterally with respect to the instrument.

In general, however, it is not necessary for the marker, a partial element of the marker or the instrument to which the marker in accordance with the invention or a partial element of the marker is attached, to be rotationally symmetrical. The marker in accordance with the invention, for example, can also be designed to be rotationally asymmetrical, i.e. it is sufficient in the sense of the invention if at least a partial area of a marker, preferably for each spatial orientation or rotation of the instrument to which the marker is attached, can be detected. If rotationally asymmetrical markers or partial elements of markers are used, then it is possible—given a suitable arrangement around the instrument of markers of different dimensions—to establish how the instrument is currently rotated or spatially orientated, on the basis of marker surfaces which may be detected when the instrument is variously rotated or orientated.

In accordance with a further aspect, the invention relates to an instrument, preferably a medical instrument, advantageously having at least one approximately rotationally symmetrical part to which a marker is attached as described above.

Advantageously, at least two markers or partial elements of markers are provided on an instrument, to increase the precision in spatial detection via light emitted or reflected by the markers. To this end, the at least two markers are advantageously offset with respect to each other by a particular distance, for example—in the case of a rotationally symmetrical instrument—spaced from each other in the axial direction or in the direction of the axis of symmetry.

In accordance with another aspect, the present invention relates to a system comprising an instrument and a marker as described above, attached to said instrument, and at least one, preferably two, cameras which serve to detect the light emitted or reflected by the marker.

An evaluation unit is advantageously provided which can determine the spatial position of the at least one marker and therefore of the instrument connected to the marker, from the information detected by the at least one camera.

In accordance with a further aspect of the invention, a method is proposed for localizing at least one marker as described above, preferably for localizing an instrument comprising at least one marker attached to it, wherein the position of at least one marker on the instrument is detected by a camera, a correction value for determining the position of the instrument is determined from the detected position of the at least one marker, and the spatial position of the instrument is determined on the basis of the correction value.

If the light emitted or reflected from a marker is detected by a camera which is not arranged on a line perpendicular to the surface of the marker, then an error with respect to the spatial position detected arises due to the oblique view, which is corrected in accordance with the invention in order to determine the correct spatial position from the data captured. To this end, an iterative method is advantageously employed which initially starts from the assumption that the determined position of the at least one marker, for example the reflection center of a ring-shaped marker, represents the correct position of the ring, which however deviates from the actual position due to a possibly oblique view, since when detecting the marker at an oblique angle, the center point of the reflective surface detected by the camera deviates from the actual center point of the marker. Proceeding from the provisional assumption that the detected position is the correct position of the marker or ring, the line of sight of the at least one, preferably two or more, detection cameras is determined for each marker or ring. A correction value for each camera and/or for each marker or ring is calculated from these lines of sight, from which virtual positions of a marker or ring are determined as the center point between the lines of sight. From this, a correction vector or shift between the position assumed to be correct and the virtual position as described above is determined. The correction vector is then subtracted from the position originally assumed to be correct, which deviates from the actual position, in order to obtain a better or at best even correct value for the correct position. Proceeding from this corrected value of the position assumed to be correct, the method is iteratively repeated until the deviation or the correction vector fall below a particular predeterminable limit value, i.e. until a predetermined accuracy is achieved.

In accordance with another aspect, the invention relates to a method for determining the spatial position of at least one marker, the spatial position of the at least one marker being iteratively determined. The iterative method presented in its main features above can also be used in general with any design of markers of arrangements of markers, to determine the spatial position of at least one marker, such as for example three markers arranged on a reference star. Thus in a first step, the light emitted—for example, actively emitted or passively reflected—by the at least one marker, preferably two, three or more markers, is optically detected by one, two or more cameras, from which the spatial position of the arrangement of markers can be approximately determined, using the knowledge of the relative arrangement of the individual markers with respect to each other, for example the geometry of the reference star. If the spatial position of an arrangement of markers is known, then the spatial position of an instrument connected to the arrangement of markers can also be determined from this. If, for example, the assumption is initially made that the individual markers are spherical or spherical-symmetrical or are spherical reflective surfaces, then the center point of the light emitted from each marker is provisionally assumed to be the center point or position of the marker. If non-spherical markers are used, then providing the individual markers are arranged on a reference star in a predetermined way, the orientation of these markers on the reference star is already known before the method is performed. Using this information in combination with the approximately determined spatial position of the arrangement of markers, for example of the reference star, and taking into account the known geometry of the markers—i.e. for example, the information that conical or cylindrical markers for example are being used—and taking into account the known position of at least one camera serving the purpose of optical detection and, as appropriate, the position of at least one light-generating element if passive reflective markers are used, then a simulation can be carried out to determine what optical signals should have been received when the spatial position of the arrangement of markers was approximately determined. If the received optical signals determined in the simulation correspond to the optical signals actually received, then the approximately determined spatial position of the arrangement of markers is correct. If, however, a deviation or difference arises between the simulated optical signals to be received and the optical signals actually received, then a correction variable can be determined from this, such as for example a difference or a three-dimensional correction vector or shift vector, by which for example a marker must be shifted or the arrangement of markers rotated, in order to obtain a better approximation or even the correct spatial position of the arrangement of markers. Once the at least one approximately determined spatial position of the arrangement of markers has been corrected or shifted, a new simulation of the optical signals to be received can be carried out using the information mentioned above, to determine whether the optical signals to be received, calculated in accordance with the new simulation, correspond better or completely with the optical signals actually received. Here again, a correction value or difference between the optical signals actually received and the simulated optical signals to be received can be determined, wherein again the correction value can be used for another simulation. This method can for example be iteratively continued until the correction value or difference falls below a predetermined admissible tolerance value for an error or until complete correspondence between the simulated and actually received optical signals is even obtained. The maximum number of iterative steps to be carried out can for example also be predetermined as a termination condition.

Advantageously, markers can be used for carrying out the method which do not exhibit spherical surfaces, such as for example two-dimensional or three-dimensional structures, for example cylindrical, conical or cubiform objects. This simplifies the production and sterilization of the markers, since it is for example relatively costly to apply reflective coatings or films to spherical surfaces, and these are furthermore relatively costly to disinfect, as opposed for example to conical or cylindrical objects. In general terms, the markers can be active, light-emitting elements, such as for example LEDs, or also passive, reflective elements or surfaces.

In accordance with another aspect, the invention relates to a computer program which performs one of the methods described above when it is loaded on a computer or is running on a computer. Furthermore, the invention also relates to a program storage medium or a computer program product comprising the aforementioned program.

In accordance with another aspect, the invention relates to a device for determining the spatial position of at least one marker using a camera and a computational unit for performing at least one of the method steps described above, wherein light-emitting elements can be provided either as markers themselves or separately from the markers, for example in a known positional relationship with respect to the at least one camera or also around an individual camera as an approximately ring-shaped element.

The invention further relates to a system comprising a device described above and at least one marker, preferably an arrangement of markers consisting of two, three or more markers, which can be detected by the at least one camera. To this end, the markers used preferably have a two-dimensional or three-dimensional structure, wherein advantageously no spherical surfaces are present. Thus, cylindrical, conical or cubiform markers can for example be used, or also markers having a different geometry, such as for example truncated conical or elliptical markers.

The invention will now be described by way of a preferred embodiment of a ring-shaped marker, and referring to the enclosed figures. There is shown:

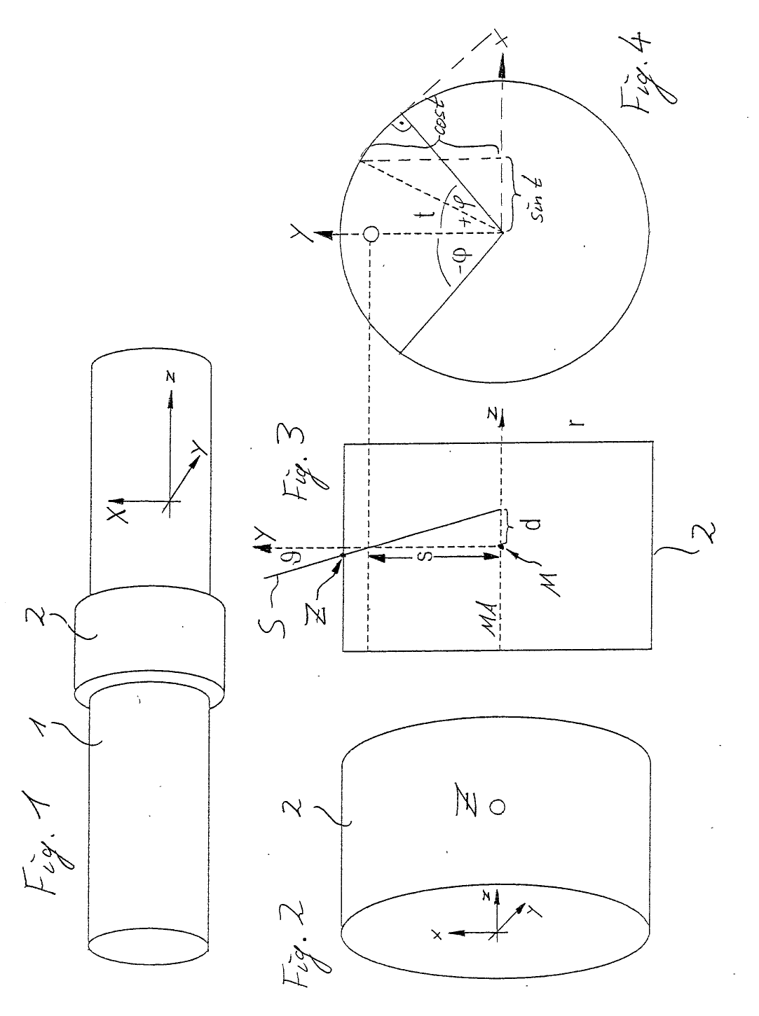

FIG. 1 an embodiment of a marker in accordance with the invention, on a cylindrical instrument;

FIG. 2 a perspective view of a marker in accordance with the invention;

FIG. 3 a cross-sectional view of the marker in accordance with the invention, in the y-z plane; and

FIG. 4 a cross-sectional view of the marker in accordance with the invention, in the x-y plane.

FIG. 1 shows a cylindrical instrument 1, such as for example the axis of a drill or screw driver which for example can be used for medical purposes. A ring-shaped marker 2 is attached in accordance with the invention to the cylindrical instrument 1 such that, irrespective of the cylindrical element 1 rotating, a surface of the marker 2 can always be detected by a camera arranged laterally with respect to the cylindrical element 1. To elucidate the following figures, an x-y-z co-ordinate system in drawn in FIG. 1.

FIG. 2 shows a perspective view of the ring-shaped marker 2 as it would be detected by a camera (not shown) which is not arranged on the line perpendicular to the surface of the marker, but looks onto the marker 2 obliquely. The reflection center Z drawn in FIG. 2 is the center point of the visible surface of the marker 2 as viewed from the camera, but which—due to the oblique view—deviates from the correct center point of the marker 2, which in the example shown in FIG. 2 is just to the right of the reflection center point Z.

FIG. 3 is a section through the marker 2 shown in FIG. 2 and represents the y-z plane. The camera looking obliquely onto the marker 2 detects the marker 2 from the direction of the line of sight S, which goes through the reflection center Z and intersects the center axis MA of the ring-shaped marker 2 at the distance d from the center point M of the centre axis MA. If the reflection centre Z detected by the camera were used as the true center point of the marker 2, and the inaccuracy due to the oblique view of the marker 2 not taken into account, then the positional error d with respect to the spatial position of the marker 2 and therefore of the instrument connected to the marker 2 would be obtained. In order to correct said positional error d, the assumption is initially made that the determined reflection center Z is in the center of the surface of the marker. If two markers 2 are attached to the instrument at a known distance from each other, then based on the assumption that the reflection centers Z1 and Z2 (not shown) for the two markers 2 are the correct reflection centers, the line of sight S and therefore the angle [symbol] can be determined, the line of sight S being shown at said angle [symbol] to the perpendicular on the surface of the marker 2.

FIG. 4 shows a section through the marker 2 in the x-y plane. If the angle to the y-axis is indicated by t, then the normal vector onto the surface of the marker 2 is given by (sin t; cos t; 0). The direction vector of the line of sight S, as shown in FIG. 3, is given by (0; cos [symbol] ; sin [symbol] ). The angle β between the normal onto the surface and the line of sight S is given by:

cos t·cos [symbol] =cos β

The local surface, which can be seen by a camera from the direction of the line of sight S, is proportional to cos β. This angle β must not exceed the critical reflection angle α (not shown) of the reflective film, which is for example about 50°. Correspondingly, the maximum value Φ which t can assume as its magnitude is defined by

cos Φ=cos α/cos

[symbol]

Overall, a camera which in FIG. 4 is arranged above the reflective ring 2 can detect a surface area starting from the angle −Φ up to the angle +Φ. In order to determine the shift d shown in FIG. 3, by which the reflection center Z is shifted from the true center point M of the center axis MA of the reflective body 2, the length s marked in FIG. 3 is determined. As can be seen in combination with FIG. 4, the length s is the focus of the circular arc which can be detected by a camera, in the angular range −Φ to +Φ. S can be calculated from:

$$s = {\int }_{- ϕ}^{ϕ} r \cdot cos t \cdot cos t \cdot cos ϑ \cdot r \cdot ⅆ t / {\int }_{- ϕ}^{ϕ} cos t \cdot cos ϑ \cdot r \cdot ⅆ t = \frac{r}{2} ( \frac{ϕ}{sin ϕ} + cos ϕ ) \vert$$

The shift or correction value is thus given by:

d=s·tan

[symbol]

This method can be performed iteratively. Thus, the correct position of the reflective ring 2 on the cylindrical body 1 can be calculated from the position of the reflection center Z detected by a camera, and thus the spatial position of the cylindrical body 1 can be determined.

1. A marker for an instrument (1), characterized in that said marker (2) is designed such that it can be arranged on at least an area of said instrument (1), along at least a partial area of a surface encircling said instrument (1).

2. The marker as set forth in claim 1, characterized in that said marker is designed such that it can be arranged continuously around said encircling surface.

3. The marker as set forth in claim 1, characterized in that said marker to be arranged along said surface encircling said instrument (1) consists of at least two marker elements separated from each other.

4. The marker as set forth in any one of the preceding claims, characterized in that said marker is rotationally symmetrical.

5. The marker as set forth in claim 4, characterized in that said marker is ring-shaped or band-shaped.

6. The marker as set forth in any one of the preceding claims, characterized in that said marker is spherical or conical.

7. The marker as set forth in any one of claims 1 to 3, characterized in that said marker is rotationally asymmetrical.

8. The marker as set forth in any one of the preceding claims, characterized in that said marker may be affixed, slid, clipped onto said instrument (1) and/or locked in place on said instrument (1).

9. The marker as set forth in any one of the preceding claims, characterized in that said marker comprises an active element and/or a passive element.

10. An instrument comprising at least one marker as set forth in any one of claims 1 to 9.

11. The instrument as set forth in claim 10, characterized in that at least two marker elements are attached to said instrument (1), preferably in the axial direction of said instrument (1), spaced away from each other.

12. The instrument as set forth in claim 10 or 11, characterized in that said instrument is substantially rotationally symmetrical or comprises a rotationally symmetrical part.

13. A system comprising an instrument as set forth in any one of claims 10 to 12, having at least one camera for detecting light emitted or reflected by the marker (2).

14. The system as set forth in claim 13, comprising an evaluation unit for determining the spatial position of said instrument (1) from the position of said at least one marker (2) detected by said least one camera.

15. A method for localizing at least one instrument as set forth in any one of claims 10 to 12, wherein the position of the reflection center (Z) of the at least two markers (2) is detected and form this, a correction value (d) for determining the spatial position of said instrument (1) connected to said at least two markers (2) is determined.

16. The method as set forth in claim 15, characterized in that said correction value (d) for the spatial position of said instrument (1) is iteratively determined.

17. A method for determining the spatial position of at least one marker (2), wherein the spatial position of said at least one marker (2) is iteratively determined.

18. The method as set forth in claim 17, wherein light emitted or reflected by said at least one marker is optically detected.

19. The method as set forth in claim 18, wherein the spatial position of an arrangement of at least two markers (2) is approximately determined from said detected light signals, using information known beforehand about the relative position of said arrangement of markers.

20. The method as set forth in claim 19, wherein a simulation of the optical signals to be received is carried out, using said approximately determined spatial position of said arrangement of markers, said orientation of the individual marker elements (2) on said arrangement of markers known beforehand and the position of the optical detection device.

21. The method as set forth in claim 20, wherein said simulated optical signals to be received are compared with the optical signals actually received, from which a correction variable is determined.

22. The method as set forth in claim 21, wherein said approximately determined spatial position of said arrangement of markers is corrected using said correction variable determined, in order to obtain a new value for said approximately determined spatial position of said arrangement of markers, and another simulation as set forth in claim 20 is carried out.

23. The method as set forth in claim 22, wherein a termination condition is predetermined for said iterative method.

24. A computer program which performs the method as set forth in any one of claims 15 to 23 when it is loaded on a computer or is running on a computer.

25. A program storage medium or computer program product comprising said computer program as set forth in claim 24.

26. A device for identifying the spatial position of at least one marker (2), comprising at least one camera and a computational unit for performing the method as set forth in any one of claims 15 to 23.

27. The device as set forth in claim 26, wherein at least one light-emitting element is provided in a defined positional relationship with respect to the at least one camera.

28. A system comprising a device as set forth in any one of claims 26 or 27 and an arrangement of at least two markers.

29. The system as set forth in claim 28, wherein said markers are formed non-spherical-symmetrically.
